(19) United States
(12) Patent Application Publication
(10) Pub. No.: US 2016/0228594 A1
(43) Pub. Date: Aug. 11, 2016

(54) ORGANIC ACID OR WEAK ACID ADDITION TO ALUM TO INHIBIT AMMONIA VOLATILIZATION FROM POULTRY LITTER

(71) Applicant: KaMin LLC, Macon, GA (US)

(72) Inventor: Richard Douglas Carter, Macon, GA (US)

(21) Appl. No.: 15/019,936

(22) Filed: Feb. 9, 2016

Related U.S. Application Data
(60) Provisional application No. 62/114,190, filed on Feb. 10, 2015.

(51) Int. Cl. A61L 9/01

(52) U.S. Cl. CPC A61L 9/01

(57) ABSTRACT

Improved compositions are disclosed that inhibit ammonia volatilization from poultry litter and other animal enclosures. In some variations, a composition for use in reducing ammonia generation in animal enclosures comprises metal sulfate, an organic acid with pKa of about 3 or higher, and optionally a buffer. In some variations, a composition for use in reducing ammonia generation in animal enclosures comprises metal sulfate, an inorganic acid with pKa of about 1 or higher, and a buffer. A method of reducing ammonia concentration in an animal enclosure (e.g., a chicken house) includes introducing any of the compositions described herein. In some embodiments, the animal enclosure is a chicken house. The ammonia concentration may be maintained below 50 ppm NH₃, preferably below about 25 ppm NH₃.

### PRIORITY DATA

This patent application is a non-provisional application claiming priority to U.S. Provisional Patent App. No. 62/114,190, filed on Feb. 10, 2015, which is hereby incorporated by reference herein.

### FIELD OF THE DISCLOSURE

The present disclosure generally relates to control of ammonia that is volatilized from animal waste products, and conversion of ammonia to land fertilizers.

### BACKGROUND OF THE DISCLOSURE

Animal litter or manure, particularly from farm animals such as poultry and pigs, contains ammonia and phosphates. The ammonia given off into the atmosphere adversely affects farm workers and even the animals themselves. When large amounts of ammonia are present in the atmosphere of animal enclosures, the result is lower weight gain and higher mortality rates for the animals.

The nature of a chicken house is such that a strong amount of urea and other compounds that break down into ammonia are generated. The ammonia is unhealthy to the chickens and the people that work in these conditions. For people, there are regulatory limits on exposure that require that either breathing apparatus is worn or strong ventilation is installed. For chickens, studies have shown that high levels of ammonia retard growth and can cause blindness, respiratory issues and can even cause burns on the young birds' feet. This condition causes a decrease in output in these farms but also creates growing conditions that are cruel. In order to deal with this, fans are installed in the houses to remove the ammonia but in the winter this can create other problems including high energy usage, and the heaters must also work to keep the animals warm.

Alum, such as aluminum sulfate, has been used to reduce the pH, and thus the ammonia generation, of manure and animal bedding material. Alum, either in solid or liquid form, will lower the pH of the manure by hydrolysis and will convert ammonia to ammonium ions. Ammonium ions will react with sulfates to form ammonium sulfate which will precipitate in the litter, reducing ammonia emissions; the ammonium sulfate is also a water-soluble nitrogen fertilizer and can be used as such after the litter is removed. The use of alum for this purpose has an added advantage in that the soluble phosphates present precipitate in the presence of aluminum and thus the soluble phosphate content of manure is also reduced by this treatment. Soluble phosphates are known to seep into ground water or are carried in surface runoff water.

Several years ago alum was introduced as a floor treatment in poultry houses as it reacts readily with the free ammonia precipitating a white ammonia sulfate. This reduces the ammonia level at its source. Unfortunately, the speed at which the alum dissolves is slow, so alum has been coupled with the use of sulfuric acid (a feed chemical to make alum) which allows the reaction (precipitation) to occur at a much faster rate initially due to the free acid. For example, Pure7™ (Affinity Chemical, Dallas, Tex.) is a product on the market that is used to control the amount of ammonia that is volatilized in a chicken house.

In U.S. Pat. No. 5,622,697, a method for inhibiting ammonia volatilization in animal manure, particularly poultry litter for long time periods (3-6 weeks) is disclosed. The method comprises the steps of adding alum (aluminum sulfate) to poultry litter (comprising poultry manure, bedding material, spilled food and feathers) and mixing. Alum is added in sufficient quantities to reduce the pH of alum/poultry litter base composition. Other patents describing conventional approaches (alum or alum with sulfuric acid) include U.S. Pat. Nos. 5,914,104, 5,961,968, 5,928,403, and 6,468,518.

Unfortunately, as described in the above patents, the amount of acid needed to initiate the reaction is around 7-9%. This amount of sulfuric acid causes the pH of the liquid being transported to the houses and being applied to be well below 2.0. Materials with pH below 2.0 are considered hazardous (40 CFR §261.22) and as such farmers must take special precautions when using it. If any spills occur, the material must be handled under Department of Transportation hazardous materials rules.

In view of the aforementioned needs in the art, there remains a need for improved compositions that inhibit ammonia volatilization from poultry litter and other animal enclosures.

### SUMMARY OF THE DISCLOSURE

In some variations, the present disclosure provides a composition for use in reducing ammonia generation in animal enclosures, the composition comprising metal sulfate and from about 0.01 wt % to about 25 wt % organic acid or a combination of organic acids (on a dry basis and based on the metal sulfate in non-hydrated form), wherein the organic acid or a combination of organic acids has an average pKa of about 3 or higher.

In some embodiments, the composition comprises from about 1 wt % to about 15 wt % organic acid or a combination of organic acids.

In various embodiments, the organic acid or a combination of organic acids has an average pKa of about 3.5 or higher, about 4 or higher, or about 4.5 or higher.

The organic acid or a combination of organic acids may be selected from the group consisting of acetic acid, acetoacetic acid, citric acid, formic acid, lactic acid, ascorbic acid, benzoic acid, propionic acid, 3-hydroxypropanoic acid, carbonic acid, glycolic acid, glyoxylic acid, glyceric acid, acrylic acid, adipic acid, malonic acid, methymalonic acid, succinic acid, malic acid, tartaric acid, itaconic acid, mesaconic acid, glutaric acid, and combinations or salts thereof In certain embodiments, the organic acid or a combination of organic acids includes acetic acid.

Optionally, the composition further comprises an inorganic acid having a pKa of about 3 or higher. In some embodiments, the composition does not include any acids having a pKa of less than 2 or less than 3.

Other variations provide a composition for use in reducing ammonia generation in animal enclosures, the composition comprising metal sulfate and from about 0.01 wt % to about 25 wt % inorganic acid or a combination of inorganic acids (on a dry basis and based on the metal sulfate in non-hydrated form), wherein the inorganic acid or a combination of inorganic acids has an average pKa of about 1 or higher.

In some embodiments, the composition comprises from about 1 wt % to about 15 wt % inorganic acid or a combination of inorganic acids.

The inorganic acid or a combination of inorganic acids may have an average pKa of about 2 or higher, about 3 or higher, or about 4 or higher.

The inorganic acid or a combination of inorganic acids may be selected from the group consisting of phosphoric acid, hydrogen peroxide, nitrous acid, hydrazoic acid, and combinations or salts thereof.

Some variations provide a composition for use in reducing ammonia generation in animal enclosures, the composition comprising metal sulfate, from about 0.01 wt % to about 25 wt % organic acid or a combination of organic acids (on a dry basis and based on the metal sulfate in non-hydrated form), and an effective amount of a buffer, wherein the organic acid or a combination of organic acids has an average pKa of about 3 or higher.

Some variations provide a composition for use in reducing ammonia generation in animal enclosures, the composition comprising metal sulfate, from about 0.01 wt % to about 25 wt % inorganic acid or a combination of inorganic acids (on a dry basis and based on the metal sulfate in non-hydrated form), and an effective amount of a buffer, wherein the inorganic acid or a combination of inorganic acids has an average pKa of about 1 or higher.

Some variations provide a composition for use in reducing ammonia generation in animal enclosures, the composition comprising metal sulfate and from about 0.01 wt % to about 25 wt % of a combination of organic and inorganic acids (on a dry basis and based on the metal sulfate in non-hydrated form), and optionally an effective amount of a buffer, wherein the combination of organic and inorganic acids has an average pKa of about 3 or higher.

The buffer is preferably designed to maintain a pH above 2.0 when the composition is in an aqueous solution. For example, the buffer may be designed, selected, or configured to maintain solution pH at or about 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0 or higher.

In some embodiments, the composition is present in an aqueous solution with a solution pH higher than 2.0, such as about 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0 or higher.

The metal sulfate comprises or consists essentially of aluminum sulfate, in some embodiments. The aluminum sulfate, when combined with ammonia, is typically converted at least in part to ammonium aluminum sulfate.

Some variations provide a method of reducing ammonia concentration in an animal enclosure, the method including introducing, to the animal enclosure, any one or more of the compositions described herein. In some embodiments, the animal enclosure is a chicken house. The ammonia concentration is maintained below about 50 ppm NH₃, preferably below about 25 ppm, and more preferably about 10 ppm or below.

### DETAILED DESCRIPTION OF EMBODIMENTS OF THE DISCLOSURE

Certain embodiments of the present disclosure (including compositions, methods, and systems) will now be further described in more detail, in a manner that enables the claimed invention so that a person of ordinary skill in this art can make and use the present disclosure.

Unless otherwise indicated, all numbers expressing reaction conditions, concentrations, yields, and so forth used in the specification and claims are to be understood as being modified in all instances by the term “about.” Accordingly, unless indicated to the contrary, the numerical parameters set forth in the following specification and attached claims are approximations that may vary depending at least upon the specific analytical technique. Any numerical value inherently contains certain errors necessarily resulting from the standard deviation found in its respective testing measurements.

As used in this specification and the appended claims, the singular forms “a,” “an,” and “the” include plural referents unless the context clearly indicates otherwise. Unless defined otherwise, all technical and scientific terms used herein have the same meaning as is commonly understood by one of ordinary skill in the art to which this disclosure belongs. If a definition set forth in this section is contrary to or otherwise inconsistent with a definition set forth in patents, published patent applications, and other publications that are incorporated by reference, the definition set forth in this specification prevails over the definition that is incorporated herein by reference.

The term “comprising,” which is synonymous with “including,” “containing,” or “characterized by” is inclusive or open-ended and does not exclude additional, unrecited elements or method steps. “Comprising” is a term of art used in claim language which means that the named claim elements are essential, but other claim elements may be added and still form a construct within the scope of the claim.

As used herein, the phase “consisting of” excludes any element, step, or ingredient not specified in the claim. When the phrase “consists of” (or variations thereof) appears in a clause of the body of a claim, rather than immediately following the preamble, it limits only the element set forth in that clause; other elements are not excluded from the claim as a whole. As used herein, the phase “consisting essentially of” limits the scope of a claim to the specified elements or method steps, plus those that do not materially affect the basis and novel characteristic(s) of the claimed subject matter.

With respect to the terms “comprising,” “consisting of,” and “consisting essentially of,” where one of these three terms is used herein, the presently disclosed and claimed subject matter may include the use of either of the other two terms. Thus in some embodiments not otherwise explicitly recited, any instance of “comprising” may be replaced by “consisting of” or, alternatively, by “consisting essentially of.”

This disclosure is predicated on the surprising discovery of a safer alternative to conventional compositions described in the Background. In particular, it has been unexpectedly found that weak acids, such as (but not limited to) acetic acid, may be introduced to provide the desired initial acidity, while maintaining a pH above 2.0 so that the composition is considered to be non-hazardous. Despite the higher pH, the composition has been experimentally found to have the same effectiveness in terms of reaction speed and reduction of ammonia. Other weak acids could be used to adjust pH into a safe range, as could common salts of the same acid, leveraging the common ion effect. All references herein to acids should be construed, in certain embodiments, as references to salts of these acids.

“Alum” in this disclosure refers to aluminum sulfate, which may be utilized to react with free ammonia, precipitating a white ammonia sulfate. Solid or liquid alum will lower the pH of the manure, converting ammonia (NH₃) to ammonium (NH₄⁺). The ammonium will combine with sulfate (SO₄²⁻) to form ammonium sulfate (NH₄)₂SO₄, which is a water-soluble nitrogen fertilizer.

Normally, dry alum can require up to two weeks to absorb sufficient water from the air or surrounding litter to initiate the reduction of the pH of litter and thereby reduce ammonia production. Alum can be mixed with water and sprayed onto manure in an effort to accelerate its function, but this may require additional handling and it reduces the flexibility of the application timing with respect to the placement of chicks, for example, in an enclosure. The addition of water also can introduce a detrimental effect on the humidity of the house and the litter.

In this disclosure, an organic acid or an inorganic acid is added to alum, or another metal salt, and then applied in the animal enclosure (e.g., a chicken house). In some embodiments, the composition comprises, on a dry basis, from about 75 wt % to about 99.9 wt % metal sulfate (based on its non-hydrated form), and from about 0.1 wt % to about 25 wt % organic acid or a combination of organic acids, wherein the organic acid or a combination of organic acids has an average pKa of about 3 or higher.

The combination of the composition and water (or a liquid containing water) results in aqueous solution, which should be selected to maintain a solution pH of greater than 2.0. The aqueous solution may be formed at the time of forming the composition, or at a later time, such as for product transport, product application (e.g. within an animal enclosure), or product use.

In some embodiments, the acid is an organic acid, or a combination of acids that includes at least one organic acid. In some embodiments, the acid is a “weak acid,” which for purposes of this disclosure means an acid with a pKa value of about 3 or higher. In some embodiments, a combination of acids is employed with an average pKa of the acid mixture, on an acid weight basis, of about 3 or higher. Preferably, no single acid (in a combination of acids) is present with a pKa of less than 2, in some embodiments of the invention.

In various embodiments, an organic acid may be selected from the group consisting of acetic acid, acetoacetic acid, citric acid, formic acid, lactic acid, ascorbic acid, benzoic acid, propionic acid, 3-hydroxypropanoic acid, carbonic acid, glycolic acid, glyoxylic acid, glyceric acid, acrylic acid, adipic acid, malonic acid, methymalonic acid, succinic acid, malic acid, tartaric acid, itaconic acid, mesaconic acid, glutaric acid, and combinations or salts thereof.

The composition is preferably selected so that the resulting aqueous solution has a solution pH of greater than 2.0. For example, when using 7% acetic acid with aluminum sulfate, the resulting pH is about 2.4. In contrast, a product with 7% sulfuric acid (a strong acid) with aluminum sulfate has a pH of about 1.7 or less. (Note this is a logarithmic scale so this difference is quite significant.)

In some embodiments, the composition comprises, on a dry basis, from about 75 wt % to about 99.9 wt % metal sulfate (based on its non-hydrated form), and from about 0.1 wt % to about 25 wt % inorganic acid or a combination of inorganic acids, wherein the inorganic acid or a combination of inorganic acids has an average pKa of about 3 or higher.

It is understood that stronger acids can also be used, if a buffer system is designed to keep the solution pH above 2.0. In some embodiments, the cost of the acid, the needs of the farmer, or the target pH may dictate the use of a strong acid along with an appropriate salt, to create a buffer that maintains the performance of the product while increasing the pH above 2.0. In some embodiments, a strong acid having pKa >1 is utilized. In some embodiments, one or more strong acids having pKa between about 1 to 3, such as about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 are utilized.

Therefore in some embodiments, the composition comprises, on a dry basis, from about 75 wt % to about 99.9 wt % metal sulfate (based on its non-hydrated form), from about 0.1 wt % to about 25 wt % inorganic acid or a combination of inorganic acids, and an effective buffer to maintain an aqueous solution pH above 2.0.

In some embodiments, the acid or combination of acids may include an inorganic acid, such as (but not limited to) phosphoric acid, hydrogen peroxide, nitrous acid, hydrazoic acid, or combinations or salts thereof. In certain embodiments, the acid or combination of acids may include a stronger acid (pKa less than 3) in combination with weaker acids, which may be organic or inorganic acids, such that the average pKa of the acid mixture is about 3 or higher on an acid weight basis. In certain embodiments, the acid or combination of acids may include a stronger acid (pKa less than 3) along with a buffer to maintain solution pH above 2.0.

A “buffer” or “buffering agent” is a weak acid (with a conjugate base) or weak base (with a conjugate acid) used to maintain the acidity (pH) of a solution near a chosen value after the addition of another acid or base. One function of a buffer is to prevent a rapid change in pH when acids or bases are added to the solution. In this disclosure, a buffer may be utilized to customize the pH of the product for storage or shipping. As one example, using a relatively weak acid like phosphoric acid along with sodium phosphate or other phosphate counter ions will suppress the dissolution of the acid and allow use of an acid with a relatively low pKa. A buffer may also be employed with acetic acid.

A buffer may be employed to reach the target pH while still having the acid capacity (right amount of acidity) to start the desired reaction with metal salt. In some embodiments of making a buffer, an organic acid as added to reach a target potential concentration of H⁺ ions (i.e., acidity). The “potential concentration of H⁺ ions” is determined by assuming the acid is a strong acid and fully dissociates. Then, based on the desired pH, the salt of the organic acid is added to reach a concentration that forces the equilibrium to the target pH value.

As an example for the case of acetic acid (HOAc),

HOAc→H⁺+OAC⁻; [HOAc]=[H⁺] EQ. 1

so 7% HOAc by weight will give an equal concentration of H⁺ ions. But acetic acid (and generally speaking, organic acids or weak acids) does not fully dissociate. So EQ. 1 equation becomes

HOAc [symbol] H⁺+OAc⁻; Ka=[H⁺][OAc⁻]/[HOAc] EQ. 2

Using the common ion effect, one can add NaOAc (acetate salt that fully disassociates) to change the concentration of H⁺ in solution according to EQ. 2.

Alternatively, one can make the salt real time by again adding excess acid and then titrating back with a strong base (such as NaOH) to tune the solution to the right pH. In other words, a buffer is constructed by forming a conjugate base from the acid. The desired amount of acidity is selected and then a calculation is made as to how much acid can be used. Then the solution is titrated back to the right pH. This approach is an alternative to the use of the salt of the organic acid; it can be faster and lower cost while reaching the same concentrations in EQ. 2, without impacting product performance.

In some variations, the present disclosure provides a composition for use in reducing ammonia generation in animal enclosures, the composition comprising metal sulfate and from about 0.01 wt % to about 25 wt % organic acid or a combination of organic acids (on a dry basis and based on the metal sulfate in non-hydrated form), wherein the organic acid or a combination of organic acids has an average pKa of about 3 or higher.

In some embodiments, the composition comprises from about 1 wt % to about 15 wt % organic acid or a combination of organic acids.

In various embodiments, the organic acid or a combination of organic acids has an average pKa of about 3.5 or higher, about 4 or higher, or about 4.5 or higher.

The organic acid or a combination of organic acids may be selected from the group consisting of acetic acid, acetoacetic acid, citric acid, formic acid, lactic acid, ascorbic acid, benzoic acid, propionic acid, 3-hydroxypropanoic acid, carbonic acid, glycolic acid, glyoxylic acid, glyceric acid, acrylic acid, adipic acid, malonic acid, methymalonic acid, succinic acid, malic acid, tartaric acid, itaconic acid, mesaconic acid, glutaric acid, and combinations or salts thereof In certain embodiments, the organic acid or a combination of organic acids includes acetic acid.

Optionally, the composition further comprises an inorganic acid having a pKa of about 3 or higher. In some embodiments, the composition does not include any acids having a pKa of less than 3.

Other variations provide a composition for use in reducing ammonia generation in animal enclosures, the composition comprising metal sulfate and from about 0.01 wt % to about 25 wt % inorganic acid or a combination of inorganic acids (on a dry basis and based on the metal sulfate in non-hydrated form), wherein the inorganic acid or a combination of inorganic acids has an average pKa of about 1 or higher.

In some embodiments, the composition comprises from about 1 wt % to about 15 wt % inorganic acid or a combination of inorganic acids.

The inorganic acid or a combination of inorganic acids may have an average pKa of about 2 or higher, about 3 or higher, or about 4 or higher.

The inorganic acid or a combination of inorganic acids may be selected from the group consisting of phosphoric acid, hydrogen peroxide, nitrous acid, hydrazoic acid, and combinations or salts thereof.

Some variations provide a composition for use in reducing ammonia generation in animal enclosures, the composition comprising metal sulfate, from about 0.01 wt % to about 25 wt % organic acid or a combination of organic acids (on a dry basis and based on the metal sulfate in non-hydrated form), and an effective amount of a buffer, wherein the organic acid or a combination of organic acids has an average pKa of about 3 or higher.

Some variations provide a composition for use in reducing ammonia generation in animal enclosures, the composition comprising metal sulfate, from about 0.01 wt % to about 25 wt % inorganic acid or a combination of inorganic acids (on a dry basis and based on the metal sulfate in non-hydrated form), and an effective amount of a buffer, wherein the inorganic acid or a combination of inorganic acids has an average pKa of about 1 or higher.

Some variations provide a composition for use in reducing ammonia generation in animal enclosures, the composition comprising metal sulfate and from about 0.01 wt % to about 25 wt % of a combination of organic and inorganic acids (on a dry basis and based on the metal sulfate in non-hydrated form), and optionally an effective amount of a buffer, wherein the combination of organic and inorganic acids has an average pKa of about 3 or higher.

The buffer is preferably designed to maintain a pH above 2.0 when the composition is in an aqueous solution. For example, the buffer may be designed, selected, or configured to maintain solution pH at or about 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0 or higher.

In some embodiments, the composition is present in an aqueous solution with a solution pH higher than 2.0, such as about 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0 or higher.

The metal sulfate comprises or consists essentially of aluminum sulfate, in some embodiments. The aluminum sulfate, when combined with ammonia, is typically converted at least in part to ammonium aluminum sulfate.

Some variations provide a method of reducing ammonia concentration in an animal enclosure, the method including introducing, to the animal enclosure, any one or more of the compositions described herein. In some embodiments, the animal enclosure is a chicken house. The ammonia concentration is maintained below about 50 ppm NH₃, preferably below about 25 ppm, and more preferably about 10 ppm or below.

The compositions provided herein may be broadcast-applied onto animal manures, such as poultry manure or litter composition present in a growing facility, by several different methods, including, but not limited to the following; (1) by hand, (2) using a fertilizer spreader, (3) using a manure spreader, and/or (4) using a litter truck. Liquid alum may be applied using the following methods; (1) backpack sprayer, (2) boom sprayer pulled behind a truck or tractor, and/or (3) a system of stationary or moving spraying devices permanently attached to the house.

The composition may be applied periodically as needed to lower atmospheric ammonia levels suitable for maximizing animal husbandry. For most animals, this level is approximately 10-50 ppm NH₃. Therefore, the amount of ammonia present in the atmosphere of an animal enclosure should be held below about 10-50 ppm, such as below about 50, 45, 40, 35, 30, 25, 20, 15, or 10 ppm.

The reaction product(s) may be adjusted to precipitate a selected nutrient, by varying the type of acid and the composition. For example, it may be desirable to produce phosphates, nitrates, sulfates, and/or acetates so that the used chicken litter may be utilized at a later date to fertilize farm land with the selected nutrients. This is beneficial from an environmental standpoint, as well as economically to provide additional revenue to the farmer/producer.

All publications, federal regulations, patents, and patent applications cited in this specification are incorporated herein by reference in their entirety as if each publication, patent, or patent application was specifically and individually put forth herein.

In this detailed description, reference has been made to multiple embodiments of the disclosure and non-limiting examples relating to how the disclosure can be understood and practiced. Other embodiments that do not provide all of the features and advantages set forth herein may be utilized, without departing from the spirit and scope of the present disclosure. This disclosure incorporates routine experimentation and optimization of the methods and systems described herein. Such modifications and variations are considered to be within the scope of the invention defined by the claims.

Where methods and steps described above indicate certain events occurring in certain order, those of ordinary skill in the art will recognize that the ordering of certain steps may be modified and that such modifications are in accordance with the variations of the disclosure. Additionally, certain of the steps may be performed concurrently in a parallel process when possible, as well as performed sequentially.

Therefore, to the extent that there are variations of the disclosure, which are within the spirit of the disclosure or equivalents of the appended claims, it is the intent that this patent will cover those variations as well. The present disclosure shall only be limited by what is claimed.

What is claimed is:

1. A composition for use in reducing ammonia generation in animal enclosures, said composition comprising metal sulfate and from about 0.01 wt % to about 25 wt % organic acid or a combination of organic acids (on a dry basis and based on said metal sulfate in non-hydrated form), wherein said organic acid or a combination of organic acids has an average pKa of about 3 or higher.

2. The composition of claim 1, wherein said composition comprises from about 1 wt % to about 15 wt % organic acid or a combination of organic acids.

3. The composition of claim 1, wherein said organic acid or a combination of organic acids has an average pKa of about 3.5 or higher.

4. The composition of claim 3, wherein said organic acid or a combination of organic acids has an average pKa of about 4 or higher.

5. The composition of claim 4, wherein said organic acid or a combination of organic acids has an average pKa of about 4.5 or higher.

6. The composition of claim 1, wherein said organic acid or a combination of organic acids is selected from the group consisting of acetic acid, acetoacetic acid, citric acid, formic acid, lactic acid, ascorbic acid, benzoic acid, propionic acid, 3-hydroxypropanoic acid, carbonic acid, glycolic acid, glyoxylic acid, glyceric acid, acrylic acid, adipic acid, malonic acid, methymalonic acid, succinic acid, malic acid, tartaric acid, itaconic acid, mesaconic acid, glutaric acid, and combinations or salts thereof.

7. The composition of claim 6, wherein said organic acid or a combination of organic acids includes acetic acid.

8. The composition of claim 1, wherein said composition further comprises an inorganic acid having a pKa of about 3 or higher.

9. The composition of claim 1, wherein said composition does not include any acids having a pKa of less than 2.

10. The composition of claim 1, wherein said composition further comprises an effective amount of a buffer designed to maintain a pH above 2.0 when said composition is in an aqueous solution.

11. A composition for use in reducing ammonia generation in animal enclosures, said composition comprising metal sulfate and from about 0.01 wt % to about 25 wt % inorganic acid or a combination of inorganic acids (on a dry basis and based on said metal sulfate in non-hydrated form), wherein said inorganic acid or a combination of inorganic acids has an average pKa of about 1 or higher.

12. The composition of claim 11, wherein said composition comprises from about 1 wt % to about 15 wt % inorganic acid or a combination of inorganic acids.

13. The composition of claim 11, wherein said inorganic acid or a combination of inorganic acids has an average pKa of about 2 or higher.

14. The composition of claim 13, wherein said inorganic acid or a combination of inorganic acids has an average pKa of about 3 or higher.

15. The composition of claim 14, wherein said inorganic acid or a combination of inorganic acids has an average pKa of about 4 or higher.

16. The composition of claim 11, wherein said inorganic acid or a combination of inorganic acids is selected from the group consisting of phosphoric acid, hydrogen peroxide, nitrous acid, hydrazoic acid, and combinations or salts thereof.

17. The composition of claim 11, wherein said composition further comprises an organic acid having a pKa of about 3 or higher.

18. The composition of claim 11, wherein said composition further comprises an effective amount of a buffer designed to maintain a pH above 2.0 when said composition is in an aqueous solution.

19. The composition of either one of claim 1 or 11, wherein said composition is present in an aqueous solution with a solution pH higher than 2.0.

20. The composition of claim 19, wherein said solution pH is about 2.5 or higher.

21. The composition of claim 20, wherein said solution pH is about 3.0 or higher.

22. The composition of either one of claim 1 or 11, wherein said metal sulfate comprises aluminum sulfate.

23. The composition of claim 22, wherein said aluminum sulfate, when combined with ammonia, is converted at least in part to ammonium aluminum sulfate.

24. A method of reducing ammonia concentration in an animal enclosure, said method including introducing, to said animal enclosure, a composition according to either one of claim 1 or 11.

25. The method of claim 24, wherein said animal enclosure is a chicken house.

26. The method of claim 24, wherein said ammonia concentration is maintained below about 50 ppm in said animal enclosure.

27. The method of claim 26, wherein said ammonia concentration is maintained below about 25 ppm in said animal enclosure.
